(19) United States
(12) Patent Application Publication
(10) Pub. No.: US 2008/0226624 A1
(43) Pub. Date: Sep. 18, 2008

(54) Combined treatment of cancer by urokinase inhibition and a cytostatic anti-cancer agent for enhancing the anti-metastatic effect

(76) Inventors: Wolfgang Schmalix, Grobenzell (DE); Anneliese Schneider, Feldafing (DE); Buddy Setyono-Han, Utrecht (NL); Johannes Foekens, Capelle A/D Yssel (NL)

Correspondence Address: MCDERMOTT WILL & EMERY LLP, 600 13TH STREET, N.W., WASHINGTON, DC, 20005-3096, US

(21) Appl. No.: 12/073,693

(22) Filed: Mar. 7, 2008

Related U.S. Application Data
(60) Provisional application No. 60/905,300, filed on Mar. 7, 2007.

(51) Int. Cl. A61K 39/395; A61K 31/495; A61K 31/175; A61K 31/4745; A61P 35/00; A61K 31/7068; A61K 31/505; A61K 31/165

(52) U.S. Cl. 424/130.1; 514/255.01; 514/590; 514/616; 514/274; 514/283; 514/49

(57) ABSTRACT

The present invention relates to a combined treatment of cancer using a urokinase inhibitor and a cytotoxic or a cytostatic agent.

The present invention relates to a combined treatment of cancer using a urokinase inhibitor and a cytotoxic or cytostatic agent.

In general, a patient afflicted with a solid malignant tumor does not die of the primary tumor, which in the majority of cases can be surgically removed, but of the metastases which spread to different loci of the body and which also may become resistant to systemic therapy. At this stage, for most of the patients, cure cannot be reached and systemic therapy is merely palliative. For instance, in patients with early breast cancer, currently, a higher cure rate can only be achieved with the application of adjuvant systemic chemo- or endocrine-therapies immediately after surgical removal of the primary tumor, either sequentially or concurrently with radiotherapy, since a number of chemotherapeutic agents are known to enhance the effectiveness of radiotherapy. This modality is expected to eliminate occult metastases. Single-agent therapy in chemonaive patients with metastatic breast cancer has achieved overall response rates of 25-55%, which may be improved further to 35-80% with combination therapy. In this regard there has been growing interesting development of combination therapeutic modalities, which include anthracyclines, a number of agents with radiosensitizing ability (i.e. cisplatin, taxanes, irinotecan, 5-FU, gemcitabine), as well as with newly developed treatment regimens that aimed to interfere a specific cellular target. These new developments should hopefully lead to an increased cure rate of patients with early cancer, or prolonged survival with the lowest cost to quality of life in patients with metastatic disease.

Classical anticancer drugs kill cancer cells or reduce cancer proliferation, but do not specifically target invasive or metastatic processes although the fate of cancer patients is primarily determined by the extent of tumor spread. Metastasis and invasion depend on the ability of cancer cells to proteolytically degrade extracellular matrix components.

Therefore, a therapeutic approach for a better outcome of cancer patients is needed wherein both tumor cells are killed and metastasis is reduced. Further, it is desirable that adverse side effects are minimized, however, at least not increased.

We herein describe a novel therapeutic approach for the treatment of cancer comprising a combined administration of a cytostatic or cytotoxic anti-cancer agent and an urokinase (uPA) inhibitor. The combined treatment strategy involving a drug primarily inhibiting metastasis (uPA inhibitor), and a cytostatic or cytotoxic drug primarily inhibiting tumor proliferation, is a very promising approach to improve the patient outcome, in particular in metastatic disease patterns. Importantly, we have found that both drugs do not negatively affect each others action.

An essential prerequisite for the invasive and metastasis capacity of solid tumors is their ability to degrade and remodel the basement membrane and other extra cellular matrix protein structures surrounding the primary tumor and/or to penetrate the basement membrane. To accomplish this, various tumor cell associated proteolytic enzymes are involved through a complex proteolysis cascade comprising various protease systems, such as cathepsins, matrix metalloproteinases, and the urokinase system of plasminogen activation.

Although the (patho)biochemical connections have not been completely elucidated yet, the plasminogen activator urokinase (uPA) and the urokinase receptor (uPAR) play a central role. uPA mediates the proteolytic cleavage of plasminogen to give plasmin. Plasmin in turn is a protease which has a wide range of actions and is capable of directly breaking down components of the extracellular matrix such as fibrin, fibronectin, laminin and the protein skeleton of proteoglycans. In addition, plasmin can activate “latent” metalloproteases and the inactive proenzyme of uPA, pro-uPA.

The abundant evidence in the literature demonstrating that the urokinase system of plasminogen activation is over expressed in a large variety of tumors, and is strongly associated with adverse clinical outcome, suggests that one of the main components of this system, the serine protease uPA, might serve as a distinct target for therapeutic intervention.

Therefore, the combination of cytotoxic anti-cancer agent with an anti-proteolytic therapy is a promising therapeutic approach to deliver additive benefits for cancer patients, in particular with regard to an enhancement of the anti-metastatic effect.

In principle, for the novel therapeutic approach any active uPA inhibitor and any cytostatic or cytotoxic anti-cancer agent can be combined. The administration can be carried out simultaneous or consecutively in any appropriate dosage scheme.

The administration both of the cytostatic or cytotoxic anti-cancer agent and the uPA inhibitor can be effected by any appropriate route, for example orally, or via infusion, for example subcutaneous, intraperitoneal, intramuscular, intracutaneous or intra-arterial. The cytostatic anti-cancer agent and the uPA inhibitor can be administered in the same way or via different routes.

Exemplary uPA inhibitors, which are suitable according to the present invention, are compounds of the general formula I or II,

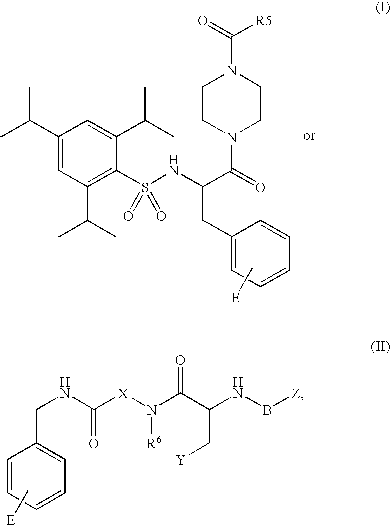

in which E is a group selected from amidine,

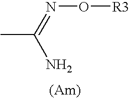

or guanidine

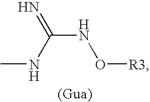

### B is —SO₂— or —CO—,

### X is —NR¹ or —CHR¹,

### Z is —R⁴, —OR⁴ or —NH—R⁴,

### Y is —OR² or NHR²,

R¹ is in each case independently —H, —C1-C6-alkyl, —C2-C6-alkenyl or —C2-C6-alkinyl, unsubstituted or substituted,

### R² is —H, —OR¹, —COR¹, —COOR¹ or CON(R¹)₂,

R³ is H, C1-C6-alkyl, C2-C6-alkenyl or C2-C6-alkinyl, unsubstituted or substituted, or —COR⁶ or —COOR⁶ or an oligo or polyalkyleneoxy radical, for example with 2-50 —C2-C4-alkyleneoxy, for example ethyleneoxy radicals,
R⁴ is H, —C1-C6-alkyl, —C2-C6-alkenyl or —C2-C6-alkinyl, unsubstituted or substituted, or a cyclic radical,
R⁵ is OR⁶, —N(R⁶)₂, —C1-C6-alkyl, —C2-C6-alkenyl or —C2-C6-alkinyl, unsubstituted or substituted, and
R⁶ is H, —C1-C6-alkyl, —C2-C6-alkenyl or —C2-C6-alkinyl, unsubstituted or substituted, or a cyclic radical,
with each cyclic radical being able to carry one or more substituents, for example selected from the group consisting of —C1-C6-alkyl, —OR⁶ (e.g. —OH or —C1-C6-alkoxy), halogen, ═O, —NO₂, —CN, —COOR⁶, —N(R⁶)₂, —NR⁶COR⁶, —NR⁶CON(R⁶)₂ and —OCOR⁶,
and it being possible for each alkyl, alkenyl or alkinyl to be straight-chained or branched and to carry one or more substituents, for example selected from the group consisting of halogen (F, Cl, Br, I), OR⁶, —OCOR⁶, —N(R⁶)₂, —NR⁶COR⁶, —COOR⁶, —NR⁶COR⁶ or a cyclic radical,
and salts or prodrugs of said compounds.

The uPA inhibitor is preferably an orally administrable agent.

Preference is further given to compounds of the general formula III

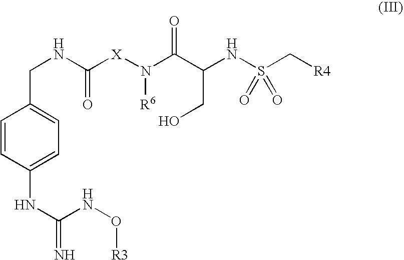

in which X, R¹, R³, R⁴ and R⁶ are defined as above,
or salts thereof.

The group E in compounds (I) and (II) is preferably located in the para position of the phenyl ring. Particular preference is given to compounds of the general formula I wherein E is (Am). Further, preferred uPA inhibitors of the formula (I) or (II) have a modified amidino or guanidine function E, preferably a hydroxyguanidino or hydroxyamidino function.

Cyclic radicals may contain one or more saturated, unsaturated or aromatic rings. Preferred examples of cyclic radicals are cycloalkyl radicals, aryl radicals, heteroaryl radicals and bicyclic radicals. Particular preference is given to mono- or bicyclic radicals. The cyclic radicals preferably contain from 4 to 30, in particular 5-10, carbon and heteroatoms as ring atoms, and also optionally one or more substitutents, as indicated above. Heterocyclic systems preferably contain one or more O, S or/and N atoms. Preference is given to those bicyclic ring systems having a —CO radical. Alkyl, alkenyl and alkynyl groups preferably contain up to 4 carbon atoms.

R¹ is preferably hydrogen or an optionally substituted C1-C4-alkyl radical, for example —CH₃ or a C1-C6-alkyl-aryl radical, so that —CO—X—NR¹ may be, for example, a glycyl, alanyl, phenylalanyl or homophenylalanyl radical.

R² is particularly preferably hydrogen or a C1-C3-alkyl radical so that Y may be, for example, an OH or O—C1-C3-alkyl radical.

R³ is particularly preferably hydrogen.

R⁵ in compounds I preferably means —NHR⁶, preferably NH(C1-C5)alkyl, unsubstituted or substituted, for example —NHC₂H₅ or —OR⁶, particularly preferably —O(C1-C3)alkyl, unsubstituted or substituted, for example ethyloxy or benzyloxy, or —O-aryl, for example phenyloxy.

R⁶ in the compounds (II) and (III) is preferably —H or C1-C3-alkyl.

Preference is given to compounds in which the structural element Z is R⁴ which is an alkyl radical having a cyclic substituent, for example an optionally substituted phenyl radical or a bicyclic radical such as, for example,

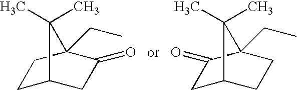

Particular preference is given to those compounds in which R4 is a substituted or unsubstituted C1-C3-alkylaryl radical, for example a benzyl radical, which may be optionally substituted in the meta or para position with halogen or/and —NO₂, said halogen being selected from the group consisting of F, Cl, Br and I, particularly preferably Cl and Br.

Most preference is given to the compounds
N-α-(2,4,6-triisopropylphenylsulfonyl)-3-hydroxyamidino-(L)-phenylalanine-4-ethoxycarbonylpiperazde (WX-671),
N-α-(2,4,6-triisopropylphenylsulfonyl)-3-hydroxyamidino-(D)-phenylalanine-4-ethoxycarbonylpiperazide,
N-α-(2,4,6-triisopropylphenylsulfonyl)-3-hydroxyamidino-(D,L)-phenylalanine-4-ethoxycarbonylpiperazide,
N-α-(2,4,6-triisopropylphenylsulfonyl)-3-hydroxyguanidino-(L)-phenylalanine-4-ethoxycarbonylpiperazide (WX-683),
N-α-(2,4,6-triisopropylphenylsulfonyl)-3-hydroxy-guanidino-(D)-phenylalanine-4-ethoxycarbonylpiperazide,
N-α-(2,4,6-triisopropylphenylsulfonyl)-3-hydroxyguanidino-(D,L)-phenylalanine-4-ethoxycarbonylpiperazide,
N-α-(2,4,6-triisopropylphenylsulfonyl)-3-hydroxy-guanidino-(L)-phenylalanine-4-ethylaminocarbonylpiperazide (WX-685),
N-α-(2,4,6-triisopropylphenylsulfonyl)-3-hydroxyguanidino-(D)-phenylalanine-4-ethylaminocarbonylpiperazide,
N-α-(2,4,6-triisopropylphenylsulfonyl)-3-hydroxyguanidino-(D,L)-phenylalanine-4-ethylaminocarbonylpiperazide,
benzylsulfonyl-(D)-Ser-Gly-(4-hydroxyguanidinobenzyl)-amide (WX-678),
4-chlorobenzylsulfonyl-(D)-Ser-N-Me-Ala-(4-hydroxyguanidinobenzyl)amide,
4-chlorobenzylsulfonyl-(D)-Ser-Gly-(4-hydroxyguanidinobenzyl)amide,
10 benzylsulfonyl-(D)-Ser-N-Me-Gly-(4-hydroxyguanidinobenzyl)amide,
4-chlorobenzylsulfonyl-(D)-Ser-Ala-(4-hydroxyguanidinobenzyl)amide.

Further preferred uPA inhibitors are N-[2-(4-Guanidino-benzenesulfonyl-amino)-ethyl]-3-hydroxy-2-phenylmethane-sulfonylamino-propionamide hydrochloride (WX-568), Bz-SO₂-(D-Ser-(Aza-Gly)-4-guanidino-benzylamide hydrochloride (WX-544), N-(4-guanidino-benzyl)-2-(3-hydroxy-2-phenylmethane-sulfonylamino-propionyl-amino)-4-phenyl-butyramide hydrochloride (WX-550), 3-nitrobenzyl-sulfonyl-(D)-Ser-Gly-(4-guanidinobenzyl)amide hydrochloride (WX-C316), benzylsulfonyl-(D)-Ser-N-Me-Gly-(4-guanidinobenzyl)amide (WX-538), N-[(4-guanidino-benzylcarbamoyl)-methyl]-3-hydroxy-2-phenylmethanesulfonylaminopropionamide (WX-508), 4-chlorobenzylsulfonyl-(D)-Ser-N-Me-Ala-(4-guanidinobenzyl)amide (WX-582), 4-chlorobenzylsulfonyl-(D)-Ser-Gly-(4-guanidinobenzyl)amide hydrochloride (WX-C340), 3-chlorobenzylsulfonyl-(D)-Ser-Gly-(4-guanidinobenzyl)amide hydrochloride (WX-C318).

A further preferred uPA inhibitor is N[alpha]-(2,4,6-triisopropylphenylsulfonyl)-3-amidino-(L)-phenylalanine 4-ethoxycarbonylpiperazide (WX-UK1).

A particularly preferred uPA inhibitor is WX-671, in particular, in form of the sulfate or hydrogen sulfate salt, for example, WX-671 • HSO₄. WX-UK1 and WX-671 are synthetic amidinophenylalanine-type small molecular weight serine-protease inhibitors that in their L-confirmation inhibit uPA and plasmin with Kᵢ values in the low- or sub-micromoles range and which suppress tumor cell invasion in vitro.

The uPA inhibitors may be in the form of salts, preferably physiologically compatible acid salts, for example salts of mineral acids, particularly preferably hydrochlorides or sulfates or hydrogensulfates, or in the form of salts of suitable organic acids, for example of organic carboxylic or sulfonic acids, such as, for example, tertates, mesylates or besylates. The uPA inhibitor may be in the form of optically pure compounds or in the form of mixtures of enatiomeres or/and diastereomeres.

For the combination therapy according to the invention, any known and approved cytotoxic or cytostatic anti-cancer agent can be used. A cytotoxic or cytostatic anti-cancer agent as defined herein is any chemical agent or drug that is selectively destructive to malignant cells and tissues. Cytotoxic or cytostatic anti-cancer agents can have different mode of actions and can be, for example, alkylating agents, platin analoga, intercalating agents, antibiotics, mitotic inhibitors, taxanes, topoisomerase inhibitors, antimetabolites or antibodies. A suitable antibody for combination with a uPA inhibitor is e.g. G250 (produced by the hybridoma DSM ACC 2526 and described in Osterweijk et al., Int. J. Cancer 38 (1986) 489-494). Other substances, such as hormones, cytokines or small molecules, for example signal transduction inhibitors or proteasome inhibitors, can be used as well.

An exemplary cytotoxic anti-cancer agent, for example, is 5-fluorouracil (5-FU), a chemotherapeutic drug widely used in the treatment of breast cancer, taxol, epirubicin or paclitaxel.

Another preferred cytotoxic anti-cancer agent is the topoisomerase-1 inhibitor, irinotecan, a water soluble Camptothecin analogue. Camptothecin originally isolated from an oriental tree, Camptotheca acuminata, is an inhibitor of topoisomerase-1 and has exhibited promising anti-tumor activity in various experimental tumors. Recently, novel camptothecin analogues, irinotecan (CPT-11) and Topotecan, have emerged as important anti-tumor agents developed over the last two decades and are presently being used for the treatment of advanced colorectal adenocarcinoma, stomach, pancreas and non-small cell lung carcinoma.

Another preferred cytotoxic agent is gemcitabine, a pyrimidine antimetabolite, 2′,2′-difluorodeoxycytidine monohydrochloride (dFdCyd). gemcitabine, an S-phase specific deoxycytidine (dCyd) nucleoside analogue, extensively modulates intracellular CTP and dCTP metabolism. In vitro studies showed that gemcitabine exposure increases the cellular concentrations and the incorporation of dFdCTP into DNA, depletes cellular CTP concentrations by inhibition of the dCTP pool. Since the activation of dFdCyd requires its phosphorylation by deoxycytidine kinase (dCK), dCK expression is essential and can predict response to gemcitabine in vivo. The incorporation of dFdCTP into DNA leads further to inhibition of DNA- and RNA-synthesis by inhibition of ribonucleotide reductase and finally lead to caspase-mediated cell death. The mechanism of action of gemcitabine, in addition to its generally favorable toxicity profile provides favorable prerequisites for combination regimens especially with compounds targeting primarily metastasis. Recently gemcitabine has been approved for the management of some cancers, i.e., pancreatic carcinoma and metastasizing breast cancer.

Further preferred cytotoxic anti-cancer agents are 5-fluorouracil (5-FU) and capecitabine. Capecitabine is an analogon of the nucleoside cytidine and is preferably used as an oral cytostatic. Capecitabine is transformed into the active agent 5-fluorouracil (5-FU). Thus, the effectivity of capecitabine is comparable to that of 5-FU. However, capecitabine is better tolerated by patients and causes markedly fewer serious side effects.

Preferred combinations according to the present invention is a combined treatment of WX-671 and 5-FU, of WX-671 and irinotecan and of WX-671 and gemcitabine as well as of WX-671 and capecitabine. Most preferred according to the invention is a combined therapy using WX-671 and gemcitabine. Further most preferred is a combined therapy using WX-671 and capecitabine.

The combination therapy according to the present invention is useful for the treatment of any kind of cancer or malignant disease. The treatment of mammary carcinoma, pancreas carcinoma, colon carcinoma, kidney cancer, lung cancer and head and neck tumors is preferred.

Particularly preferred is the treatment of cancer types which are associated with elevated expressions of urokinase, for example breast cancer.

Especially preferably, the present invention relates to the combined treatment of mammary carcinoma, pancreas carcinoma or colon carcinoma with a combination of WX-671 and gemcitabine. A combination of WX-671 and gemcitabine is preferably applied to patients having non-metastasizing pancreas cancer.

A combination of WX-671 and capecitabine is preferably used to treat patients having metastasized HER2-receptor-negative breast cancer. For the treatment, a patient is given, for example, a daily dose of WX-671 for three weeks and, additionally, a daily dose of capecitabine during the first two weeks.

According to the invention it has been found that a combination of classical cytotoxic anti-cancer agents with anti-proteolytic uPA inhibitors, in particular, with WX-671 enhances the anti-metastatic effect delivered by the cytotoxic agents alone. The spreading of cancers originating from different tissues, e.g. breast, pancreas, or colon can be reduced with uPA, in particular, WX-671 therapy. The additive anti-metastatic effects gained by combining cytotoxic therapies with uPA inhibitors such as WX-671 are achieved without significant enhancement of unwanted side effects. While uPA monotherapy, in particular, WX-671 monotherapy has a moderate effect on primary tumor growth, when applied in combination therapies, as herein described, uPA inhibitors and, in particular, WX-671 do not significantly interfere with the anti-tumor effect of cytotoxic treatments.

The administration of the cytostatic or cytotoxic anti-cancer agent and of the uPA inhibitor can be effected by any appropriate route. Preferably, the uPA inhibitor, e.g. WX-671, is administered orally. The cytostatic or cytotoxic anti-cancer agent is preferably administered intraperitoneally or i.v. The amount to be administered depends from the respective active agent and is preferably from 0.001 to 5 mg/kg body weight, preferably from 0.01 to 1 mg/kg body weight in the case of the uPA inhibitor. Administration, for example, can be effected daily, but also at larger intervals. The amount of cytostatic or cytotoxic anti-cancer agent administered is preferably from 0.001 mg/kg body weight to 100 mg/kg body weight, preferably from 0.1 mg/kg body weight to 50 mg/kg body weight. Administration is preferably effected once or twice a week.

The following figures and examples further illustrate the present invention.

### FIGURES

FIG. 1 shows the tumor weight, number of lung foci and lymph node weight associated with WX-671 or 5-FU treatment as well as the combination treatment with 5-FU and WX-671.

FIG. 1a shows the tumor load after treatment with WX-671 or 5-FU treatment as well as the combination treatment with 5-FU and WX-671.

FIG. 1b shows the thymus and the spleen weight of rats treated with WX-671 or 5-FU alone or in combination.

FIG. 1c shows the anti-metastatic effects of the treatment with WX-671 or 5-FU alone or in combination.

FIG. 1d shows the tumor load and the tumor weight of rats after treatment with WX-671 or 5-FU treatment as well as the combination treatment with 5-FU and WX-671.

FIG. 2 shows the tumor weight, number of lung foci and lymph node weight associated with WX-671 or gemcitabine treatment as well as the combination treatment with gemcitabine and WX-671.

FIG. 2a shows the tumor load after treatment with WX-671 or gemcitabine treatment as well as the combination treatment with gemcitabine and WX-671.

FIG. 2b shows the effect on primary tumor size and weight of the treatment with WX-671 or gemcitabine as well as the combination treatment with gemcitabine and WX-671.

FIG. 2c shows the anti-metastatic effects of the treatment with WX-671 or gemcitabine alone or in combination.

FIG. 3 shows the tumor weight, number of lung foci and lymph node weight associated with WX-671 or irinotecan treatment as well as the combination treatment with irinotecan and WX-671.

FIG. 3a shows the kinetics of orthotopic tumor growth associated with treatment with WX-671 or irinotecan as well as the combination treatment with irinotecan and WX-671.

FIG. 3b shows the effect on primary tumor size and weight of the treatment with WX-671 or irinotecan as well as the combination treatment with irinotecan and WX-671.

FIG. 3c shows the anti-metastatic effects of the treatment with WX-671 or irinotecan alone or in combination.

FIG. 3d shows the side effects of the treatment with WX-671 or irinotecan alone or in combination.

FIG. 3e shows the spleen and liver weight after treatment with WX-671 or irinotecan alone or in combination.

FIG. 4 shows the tumor weight, number of lung foci and lymph node weight associated with WX-671 or gemcitabine treatment as well as the combination treatment with gemcitabine and WX-671.

FIG. 4a shows the effects on pancreatic tumor weight after treatment with WX-671 or gemcitabine treatment as well as the combination treatment with gemcitabine and WX-671.

FIG. 4b shows the anti-metastatic effects of the treatment with WX-671 or gemcitabine alone or in combination.

FIGS. 4c and 4d show the effects on organs weight of the treatment with WX-671 or gemcitabine alone or in combination.

### EXAMPLES

The following examples show different combination treatment strategies aimed at inhibiting the uPA-system targeting primarily metastatic spread on the one hand and of conventional chemotherapeutic drugs targeting primarily cell proliferation on the other hand. The examples aim at the investigation of the inhibition of invasion and metastasis in different transplantable, syngeneic rat cancer models.

In particular, WX-671 has been used in combination with 5-fluorouracil (5-FU), irinotecan (IRI) or gemcitabine (GEM). Cytotoxic treatments were adjusted such a way to achieve a significant, although not complete, inhibition of tumor growth. The animal experiments were performed with the rat cancer models BN 472 and Ca20948.

The rat BN472 mammary carcinoma was established from a spontaneous breast tumor in a Brown Norwegian female rat and has been propagated by serial transplantation in syngeneic BN rats (Kort et al., J. Natl. Cancer Inst. 72:709-713, 1984). Small tumor cubes (2×2×2 mm) were prepared from a tumor grown in a donor rat and implanted orthotopically under the fat pad of the mammary gland. The tumor has a take rate of 100% and disseminates primarily to the lung to form metastatic lesions and invades the axillary lymph nodes. It has recently been shown that the BN-472 mammary also tumor expressed significant amounts of uPA, uPAR and PAI-1 as assessed by real-time RT-PCR analysis, and represents an excellent in vivo model to study the efficacy of drugs that interfere in the uPA-system of plasminogen activation.

The Ca20948 rat pancreatic adenocarcinoma, was originally developed by the azaserine induction method (described by Longnecker and Curphey in 1975). The tumor was propagated by serial transplantation in syngeneic male Lewis albino rats. For tumor implantation a tumor tissue suspension prepared from a tumor harvested from a donor rat was injected i.p. into the upper left quadrant of the peritoneum. The tumor grows intraperitoneally intimately associated with the recipient rat's pancreas. The tumor disseminates primarily to the liver to form metastatic lesions.

### Example 1

### Comparative Anti-Tumor and Anti-Metastatic Effects of WX-671, 5-FU, and the Combination of WX-671 Plus 5-FU Administrations in Brown Norway Rats Bearing BN-472 Rat Mammary Tumors

This experiment was undertaken to compare the efficacy of anti-tumor and anti-metastatic effects of monotherapy using WX-671 or 5-fluorouracil (5-FU), and of a combination of both treatments to establish possible beneficial effects.

The kinetics of tumor growth development and final tumor weights of rats treated with WX-671, two dosages of 5-FU, and combinations of WX-671 and 5-FU, with each other and with those of vehicle-treated control rats were compared.

The dosage levels were as follows:
Vehicle without anti-cancer therapy (control)
0.3 mg/kg of WX-671, daily orally (p.o.)
5-FU: 20 mg/kg, weekly intravenously (i.v.)
0.3 mg/kg of WX-671, daily p.o.+5-FU: 20 mg/kg, weekly i.v.
5-FU: 40 mg/kg, weekly i.v.
0.3 mg/kg of WX-671, daily p.o.+5-FU: 40 mg/kg, weekly i.v.

WX-671 was daily administrated orally starting 6 days after tumor inoculation, while the two dosages of 5-FU were administered intravenously once a week starting at day 6 after tumor inoculation. In all groups, treatments were well tolerated as judged from the general well-being of the animals and from the unchanged body weights compared with vehicle treated control animals.

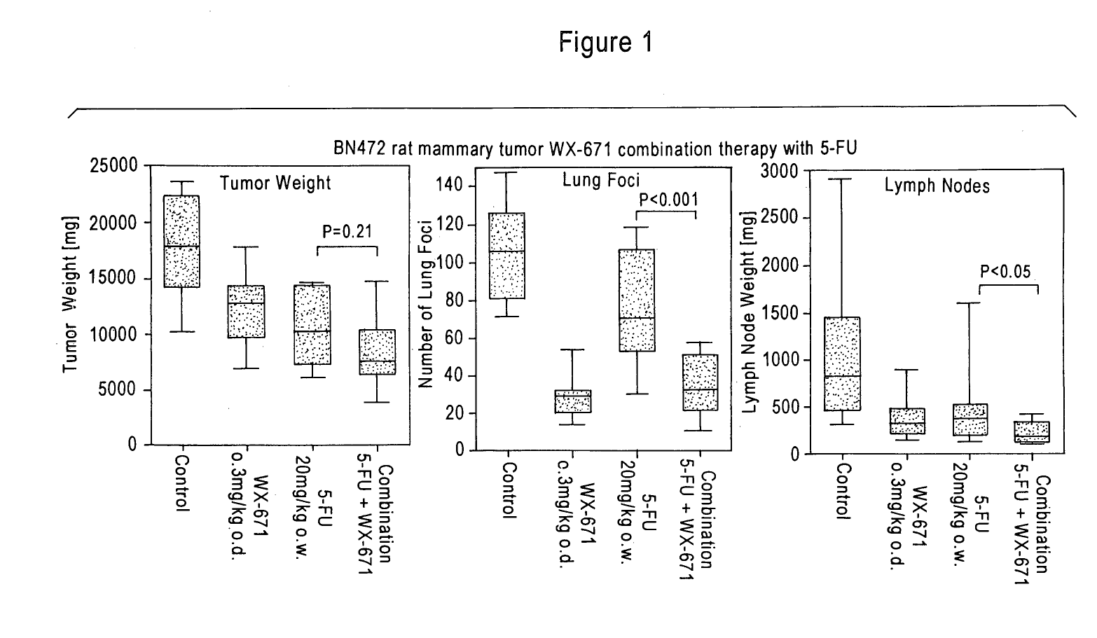

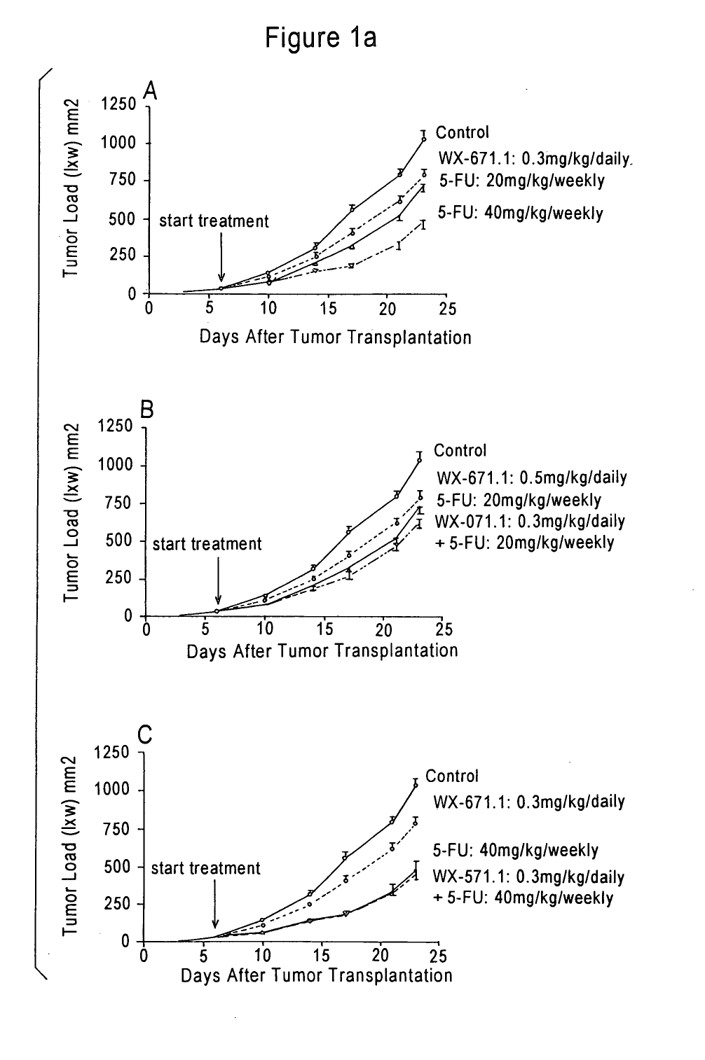

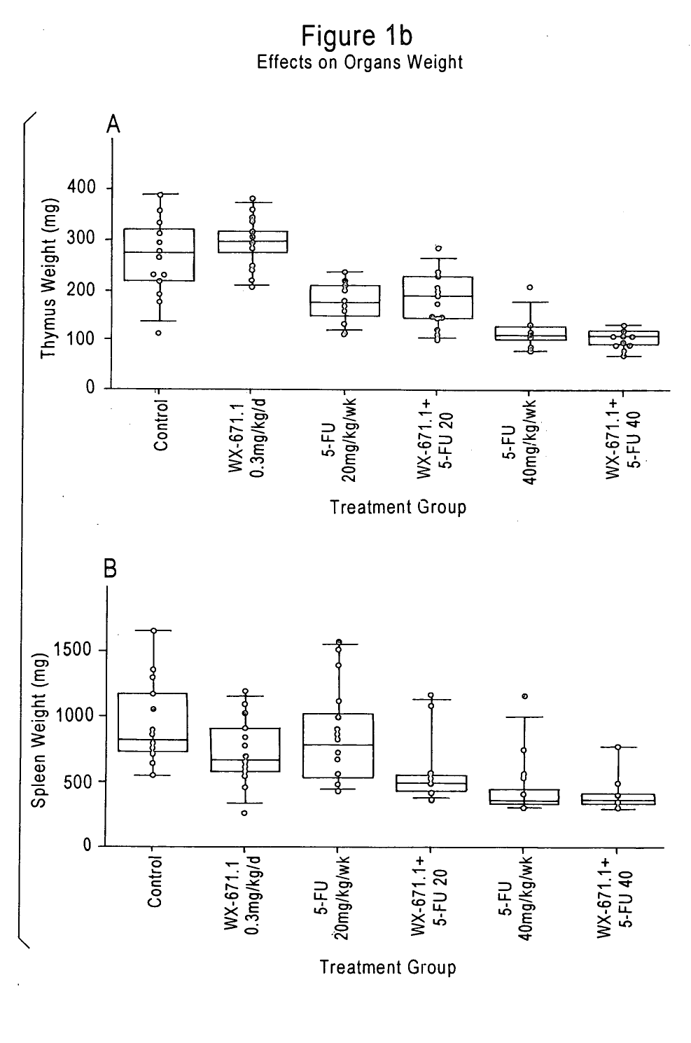

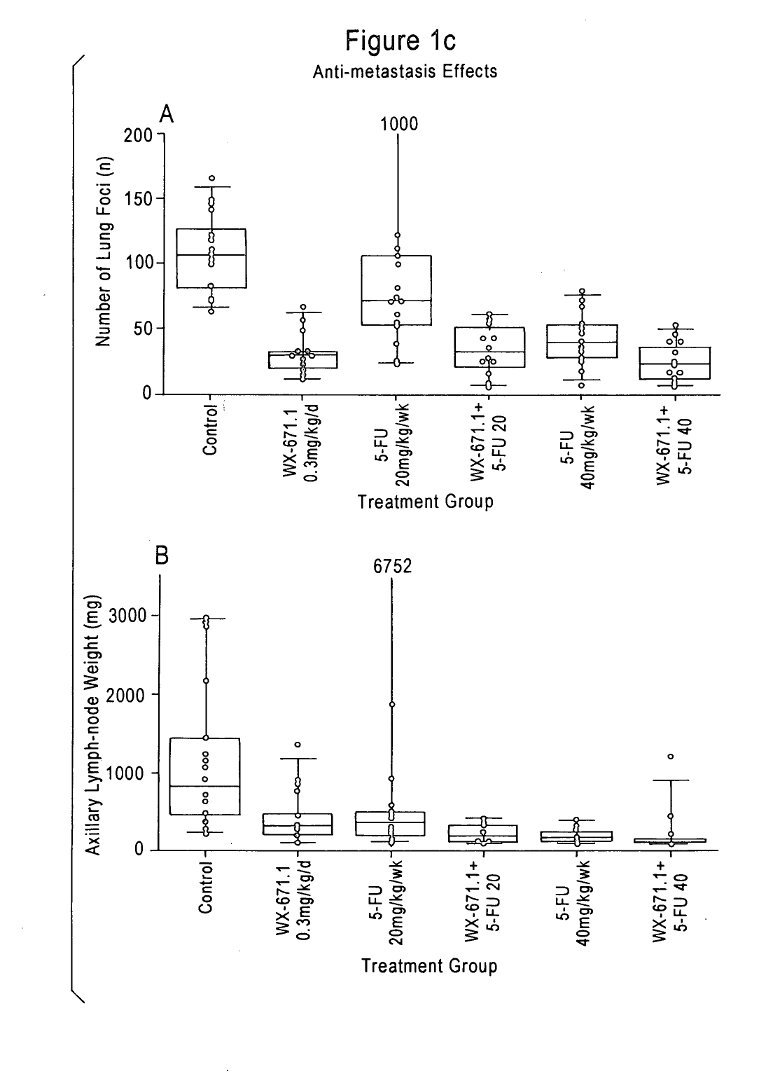

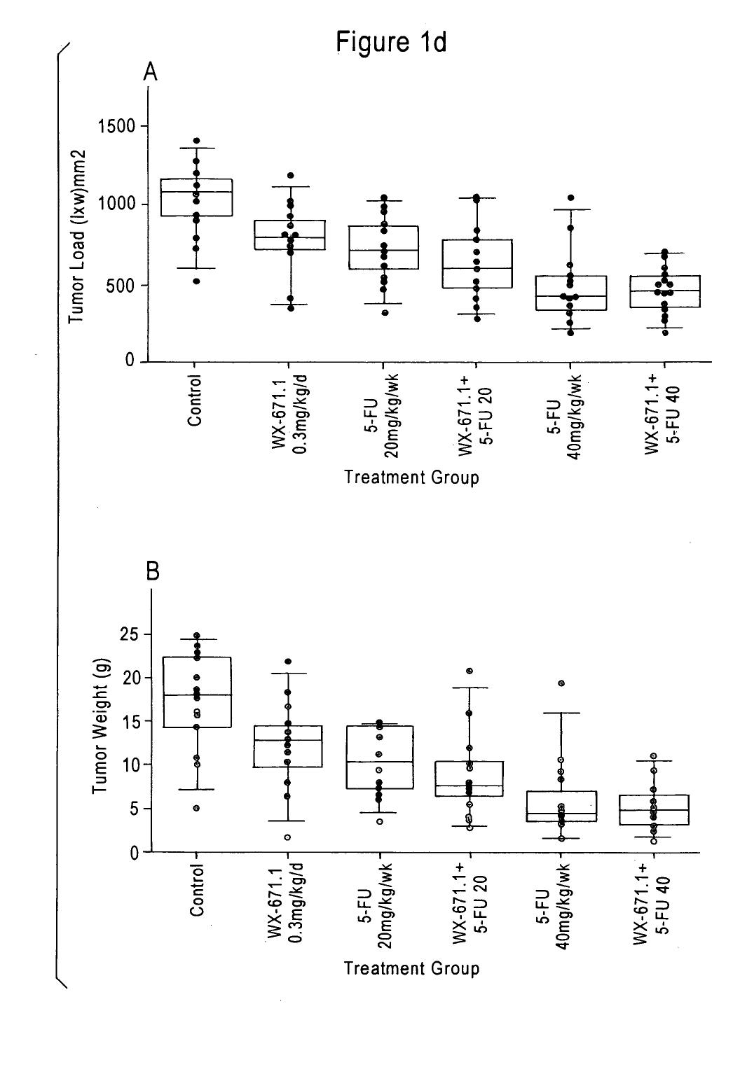

The metastatic endpoints, i.e., the number of lung foci, axillary- and intraperitoneal lymph node weights were compared, in the rats treated with WX-671, both dosages of 5-FU, or the combinations of WX-671 and 5-FU, with each other and with those of vehicle-treated control rats. The effects of treatments with WX-671, 5-FU, and the combination of WX-671 plus 5-FU on body weight and tumor load were evaluated twice weekly. The final evaluation was done at the end of the therapy period after all rats had been sacrificed. Graphical evaluations of the endpoints considered, i.e., tumor size and weight, axillary lymph-node weight, and the number of macroscopic lung foci, are depicted in the kinetics of tumor growth and/or in the graphs shown in FIGS. 1, 1a, 1 b, 1c and 1d.

Compared with 5-FU monotherapy at 20 mg/kg, the combination with WX-671 therapy improved all median endpoint parameters. Relative to the 5-FU monotherapy the combination reduced the median tumor size and weight by 16% and 17%. The metastatic parameters, lung foci counts and axillary lymph node weight, were significantly improved by 55% and 48% relative to 5-FU at 20 mg/kg alone. Thus, a combined treatment strategy involving a drug primarily inhibiting metastasis (WX-671), and a cytotoxic drug primarily inhibiting tumor proliferation (5-FU), looks very promising. Importantly, both drugs did not negatively affect each others action. The data obtained in this experiment support the assumption that the different mechanisms underlying the mainly anti-metastatic activity of WX-671, and the mainly cytotoxic activity of 5-FU, justify a combination approach of anti-metastatic with cytotoxic drugs.

### Example 2

### Comparative Anti-Tumor and Anti-Metastatic Effects of WX-671, Gemcitabine and the Combination of WX-671 Plus Gemcitabine Administrations in Brown Norwegian Rats Bearing BN-472 Rat Mammary Tumor

The objective of this experiment was to compare the anti-tumor and anti-metastatic effects of monotherapy using gemcitabine (dFdC) or WX-671, and of combinations of gemcitabine and WX-671 in the metastasizing BN-472 rat mammary carcinoma model.

The dosage levels were as follows:
Vehicle without anti-cancer therapy (control)
WX-671: 0.3 mg/kg daily p.o.
gemcitabine: 2 mg/kg, twice weekly i.p.
gemcitabine: 2 mg/kg, twice weekly i.p. plus WX-671 0.3 mg/kg daily p.o.
gemcitabine: 4 mg/kg, twice weekly i.p.
gemcitabine: 4 mg/kg, twice weekly i.p. plus WX-671 0.3 mg/kg daily p.o.

Gemcitabine was administered i.p. twice weekly, while WX-671 was administered orally, both starting 7 days after tumor inoculation.

We compared the tumor growth development and final tumor weights of rats treated with different dosages of gemcitabine, WX-671 and their combination with that of vehicle-treated control rats and with each other, as well as the metastatic endpoints, i.e., the number of lung foci and the weights of the axillary and intra peritoneal lymph-nodes. Further, possible beneficial anti-tumor and/or anti-metastatic effects of gemcitabine, WX-671 and their combined administrations were evaluated.

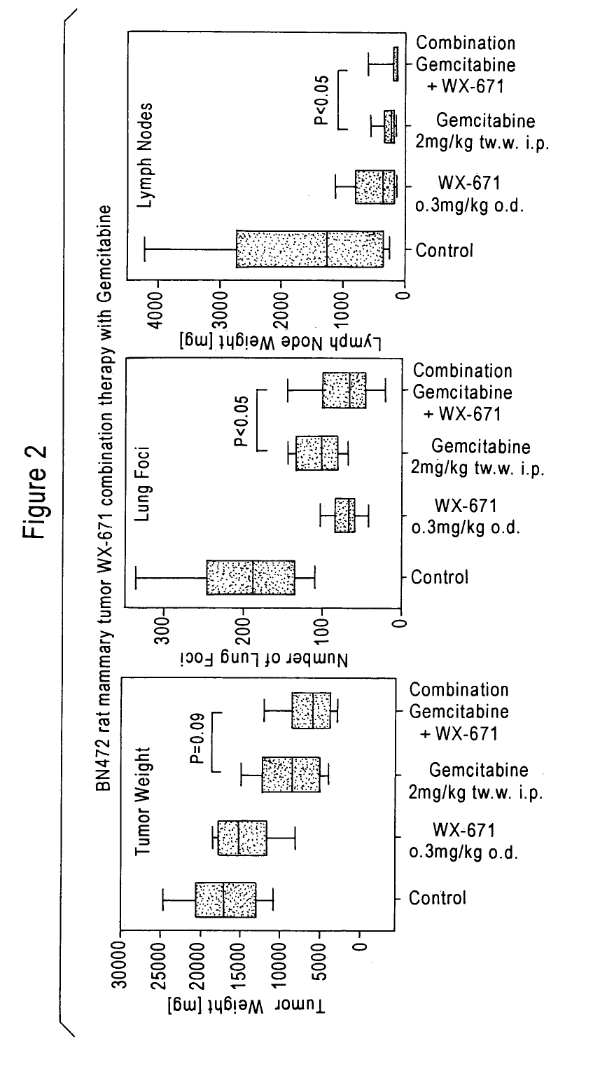

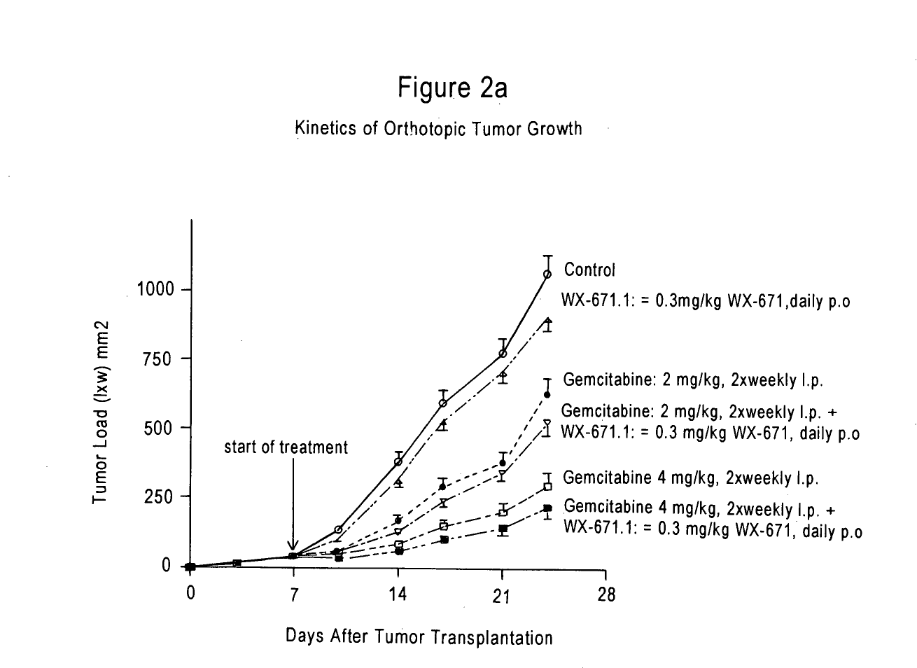

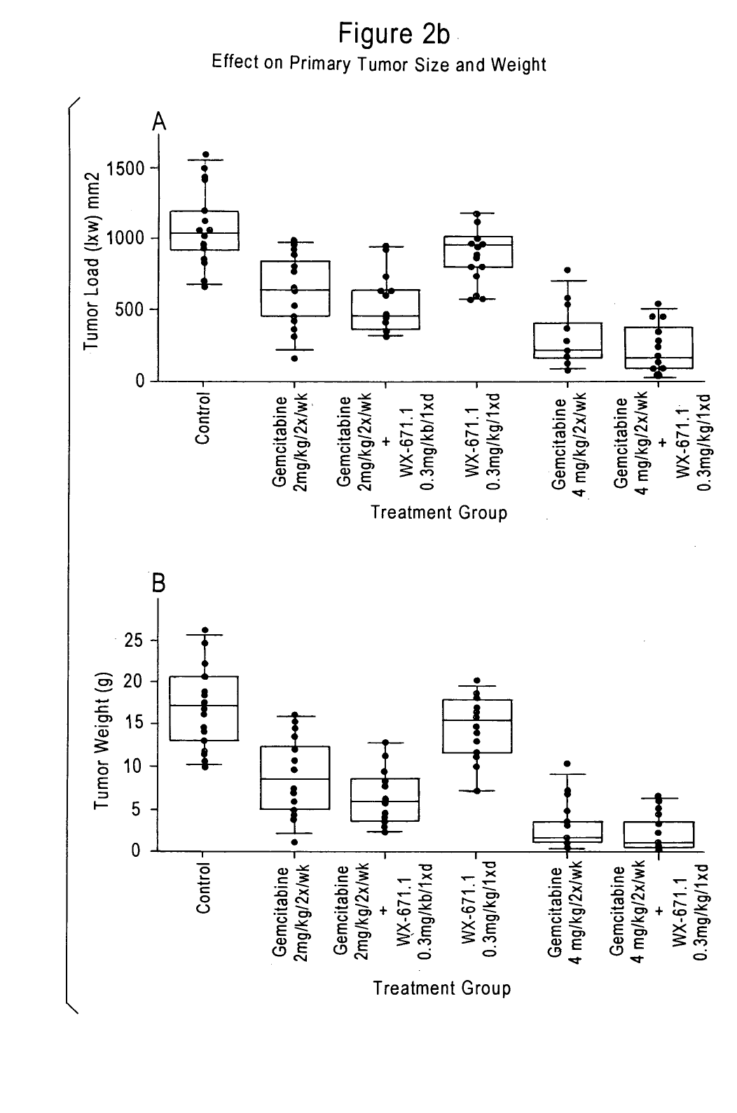

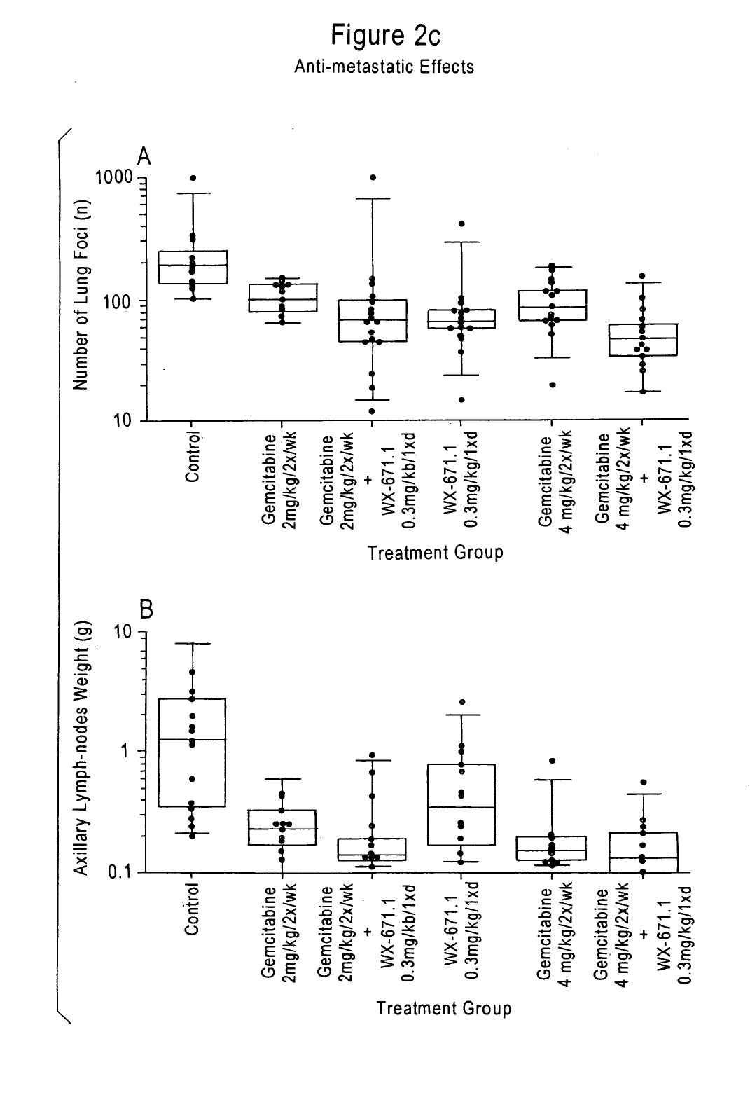

The effects of treatments with gemcitabine, on body weight and tumor load were evaluated twice weekly. The final evaluation was done at the end of the therapy period after all rats had been sacrificed. Graphical evaluations of the endpoints considered, i.e., tumor size and weight, axillary and intra peritoneal lymph-node weights, the number of macroscopic lung foci and of some organs are depicted in the kinetics of tumor growth and/or in the graphs shown in FIGS. 2, 2a, 2b and 2c.

Compared with gemcitabine monotherapy at 2 or 4 mg/kg, the combination with WX-671 therapy improved all median endpoint parameters. Relative to the gemcitabine monotherapy at both doses, the combination reduced the median tumor size and weight by approx. 30%. The metastatic parameters, lung foci counts and axillary lymph node weight, were significantly improved by 34% and 40% relative to gemcitabine at 2 mg/kg alone.

Relative to gemcitabine 4 mg/kg alone, lung foci counts were significantly reduced by 45% by combining with the WX-671 treatment schedule. The endpoint, median axillary lymph node weight, was improved by 14% by adding WX-671 therapy to the gemcitabine schedule.

The anti-tumor and anti-metastatic activity of 2 to 4 mg/kg twice weekly administrations of gemcitabine was demonstrated to be increased by concomitant administration of the uPA inhibitor WX-671. Furthermore, it can also be concluded that the combination therapy resulted in increased anti-tumor and especially anti-metastatic effects without worsening side effects. Thus, a combined treatment strategy WX-671, and gemcitabine was confirmed to be a promising approach.

### Example 3

### Comparative Study on Anti-Tumor and Anti-Metastatic Effects of the uPA-Inhibitor Prodrug WX-671, the Topoisomerase-1 Inhibitor Irinotecan (CPT-11) and Their Combination in Brown Norwegian Rats Bearing BN472 Rat Mammary Tumors

The experiment was undertaken to study the anti-tumor and anti-metastatic efficacy of monotherapy using WX-671 or irinotecan (CPT-11), and combinations of both treatments to establish possible beneficial anti-tumor and/or anti-metastatic effects upon combining WX-671 and irinotecan administrations.

We compared tumor growth development and final tumor weights of rats treated with the WX-671, with different dosages of irinotecan and with the combinations of both drugs, the metastatic endpoints, i.e., the number of lung foci as well as of the weights of the axillary and intraperitoneal lymph-nodes

The dosage levels were as follows.
Vehicle without anti-cancer therapy (control)
0.3 mg/kg of WX-671, daily p.o.
0.3 mg/kg of WX-671, daily p.o. plus irinotecan at 2 mg/kg, daily i.p.
0.3 mg/kg of WX-671, daily p.o. plus irinotecan at 6 mg/kg, daily i.p.
irinotecan at 2 mg/kg, daily i.p.
irinotecan at 6 mg/kg, daily i.p.

WX-671 was administered p.o. daily starting 3 days after tumor inoculation while irinotecan was administered i.p. daily starting 7 days after tumor inoculation. Daily observations during the treatment period indicated that both drugs were generally well tolerated.

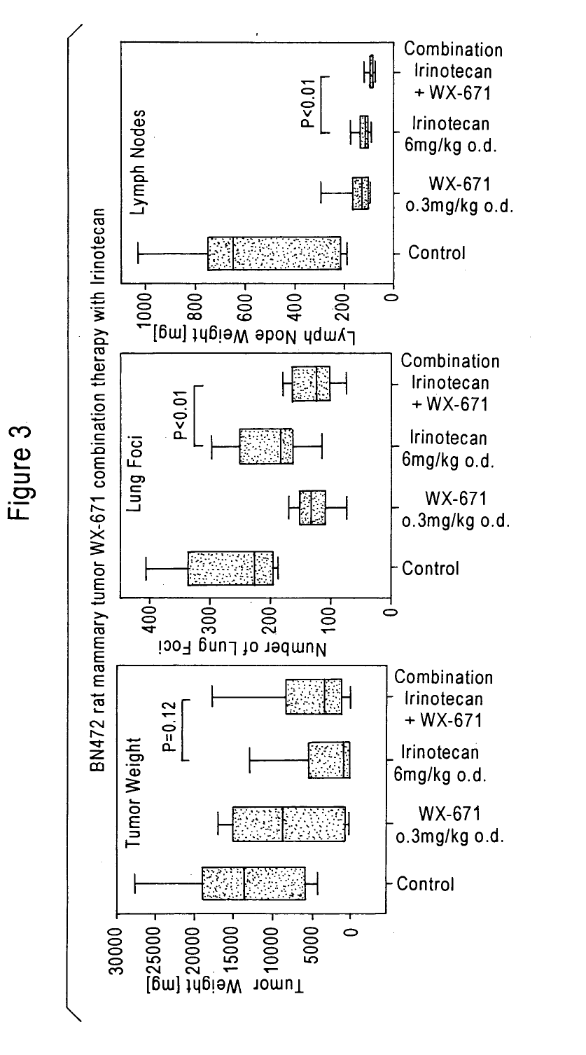

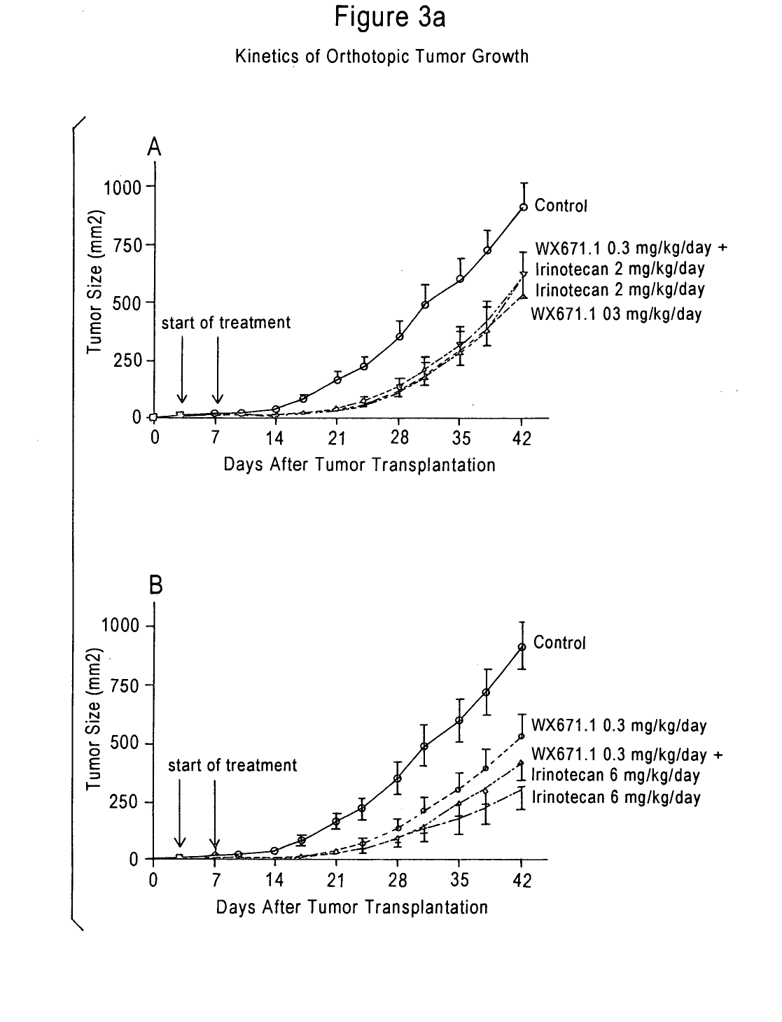

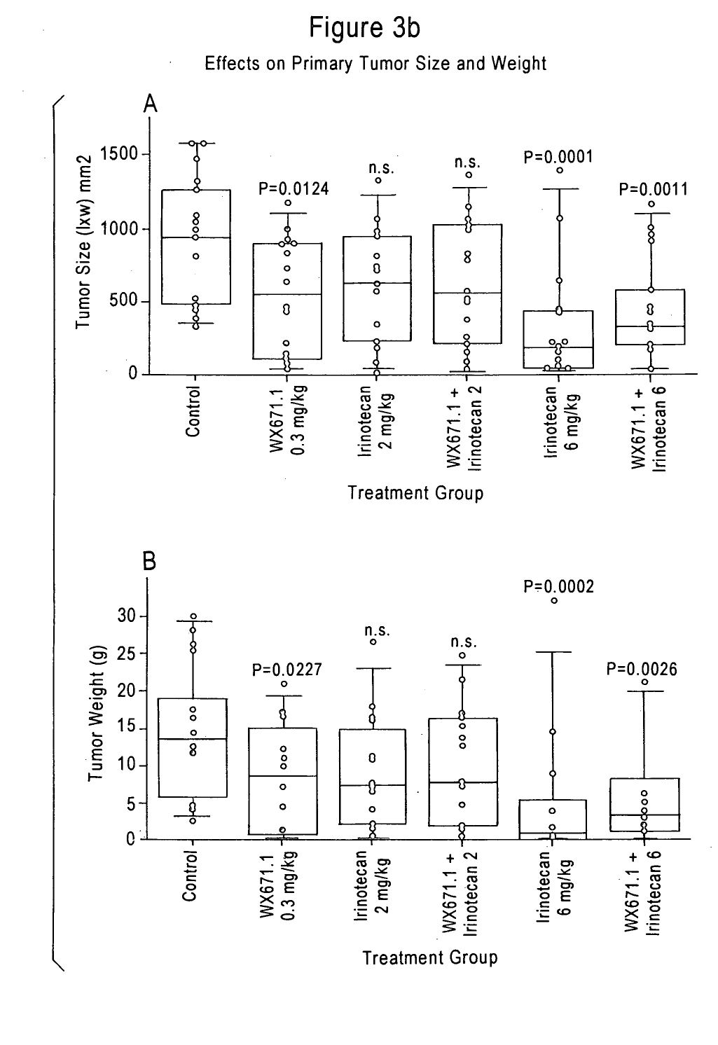

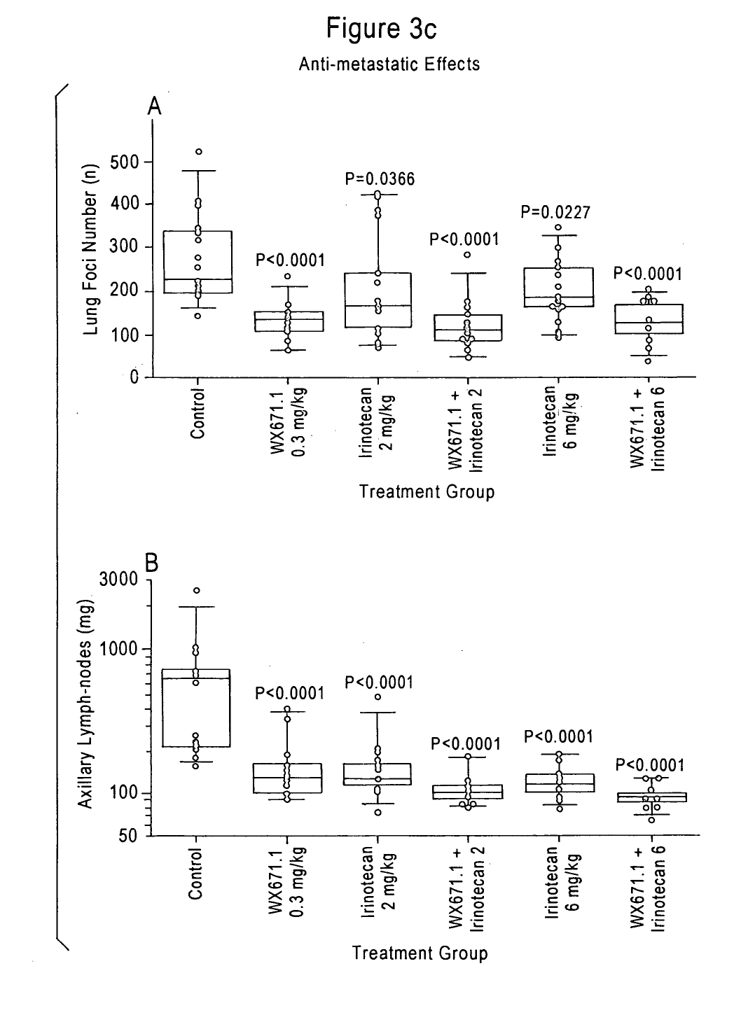

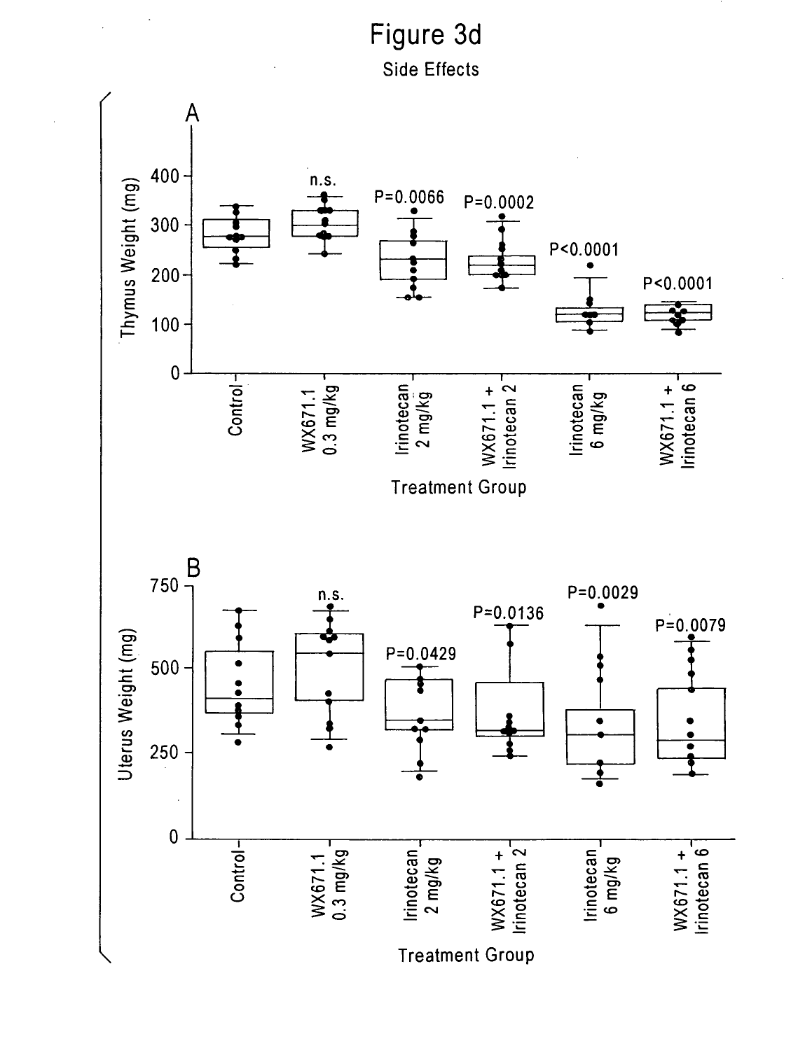

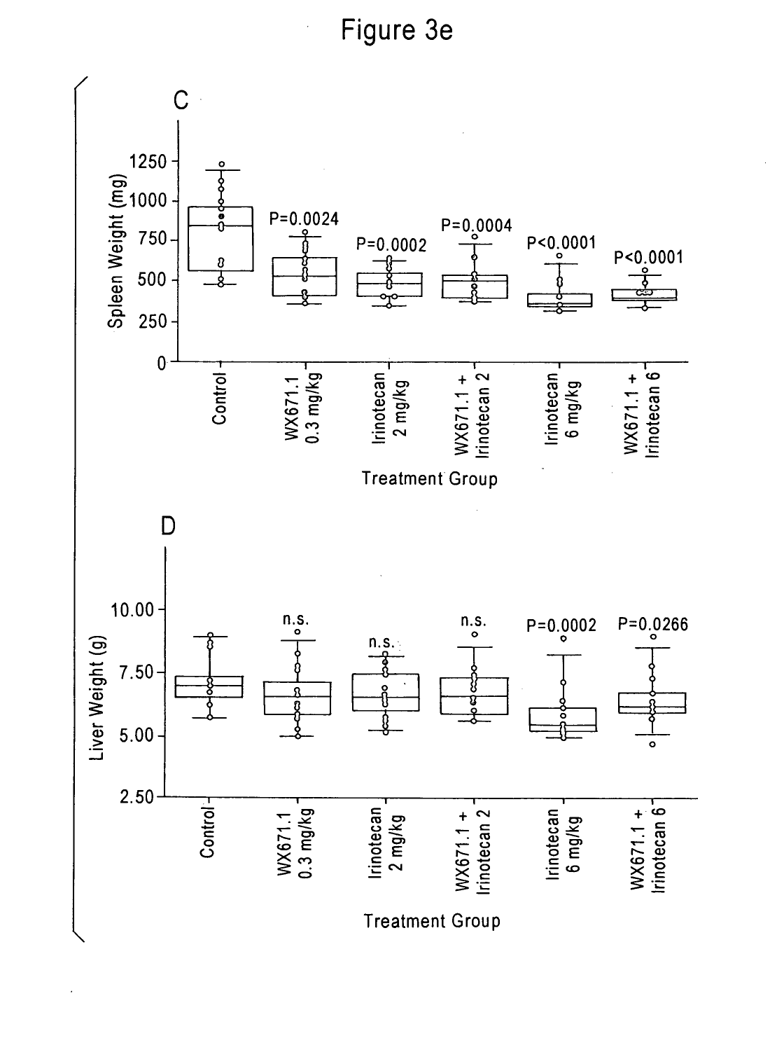

The effects of treatments on body weight and tumor load were evaluated twice weekly. The final evaluation was done at the end of the therapy period after all rats had been sacrificed. Graphical evaluations of the endpoints considered, i.e., tumor size and weight, axillary lymph-node weights, the number of macroscopic lung foci, and the weights of the thymus, uterus, spleen and liver, are depicted in FIGS. 3, 3a, 3b, 3c, 3d and 3e.

Daily administration of WX-671 as mono-therapy at 0.3 mg/kg of WX-671 resulted in significant anti-tumor and anti-metastatic effects. Daily administration of irinotecan as mono-therapy resulted in inhibition of tumor growth. At both doses of irinotecan, the number of lung foci, as well as the axillary lymph-nodes weight were significantly inhibited. The combination therapy of irinotecan plus WX-671 yielded additional reduction of metastatic endpoints in particular regarding the number of metastatic lesions in the lung. In conclusion, in the BN-472 tumor model adding-on anti-proteolytic WX-671 therapy to cytotoxic irinotecan therapy reduced the extent and severity of metastatic endpoints. No additivity was noted in the combination arms of the study relative to cytotoxic mono-treatments regarding unwanted side effects typical for classical cytotoxic anti-tumor therapy.

### Example 4

### Comparative Anti-Tumor and Anti-Metastatic Effects of WX-671, Gemcitabine and the Combination of WX-671 Plus Gemcitabine in Lewis Rats Bearing the CA-20948 Rat Pancreatic Adenocarcinoma

This experiment was undertaken to study the anti-tumor and anti-metastatic effects of the combination of WX-671 and gemcitabine compared with WX-671 and gemcitabine alone in Lewis rats bearing orthotopically transplanted CA-20948 pancreatic tumors.

The final tumor weights (pancreas with tumors) of rats treated with WX-671 or 1 and 2 mg/kg of gemcitabine, and their combinations was compared with that of vehicle-treated control rats and with each other, in addition, the metastatic endpoints, i.e., the number of liver foci in the various treatment groups were compared. We evaluated possible dose-dependent anti-tumor and/or anti-metastatic effects of gemcitabine combinations with WX-671.

The dosage levels were as follows.
Vehicle without anti-cancer therapy (control)
0.3 mg/kg of WX-671, daily p.o.
gemcitabine: 1 mg/kg, 2×/week i.p.
0.3 mg/kg of WX-671, daily p.o. plus gemcitabine 1 mg/kg, 2×/week i.p.
gemcitabine: 2 mg/kg, 2×/week i.p.
0.3 mg/kg of WX-671, daily p.o. plus gemcitabine: 2 mg/kg, 2×/week i.p.

WX-671 was administered orally daily, while the dosages of gemcitabine were administrated by i.p. injections twice weekly. All treatment modes were started 4 days after tumor inoculation. Daily observations during the whole treatment period of 20 to 21 days showed that administrations of the described dosages of either WX-671, gemcitabine or the combination of WX-671 and gemcitabine, the drugs were generally well tolerated.

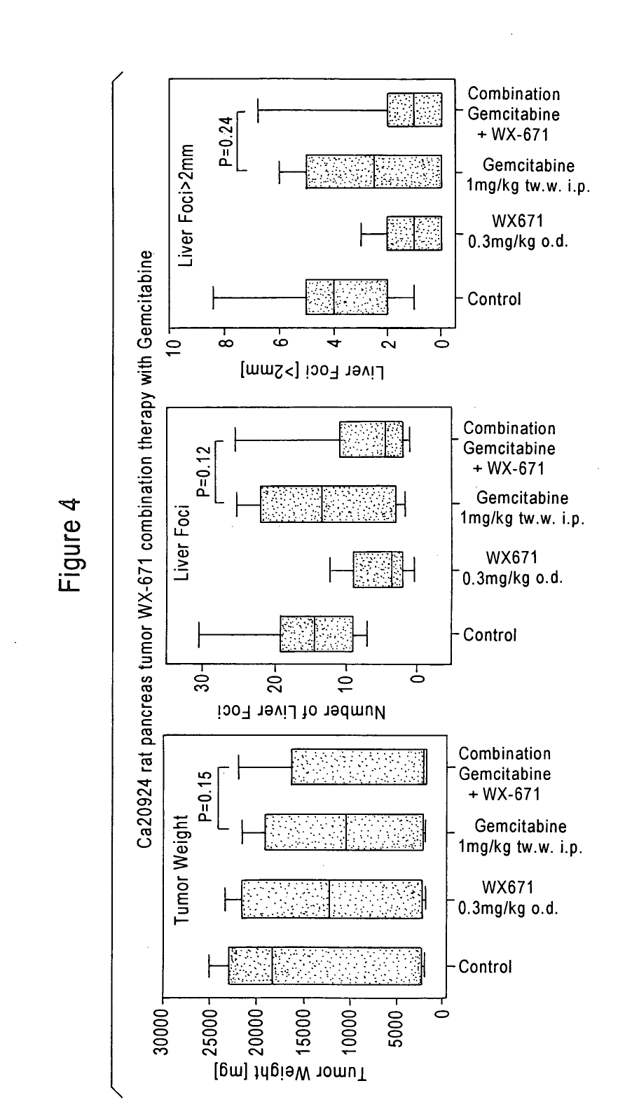

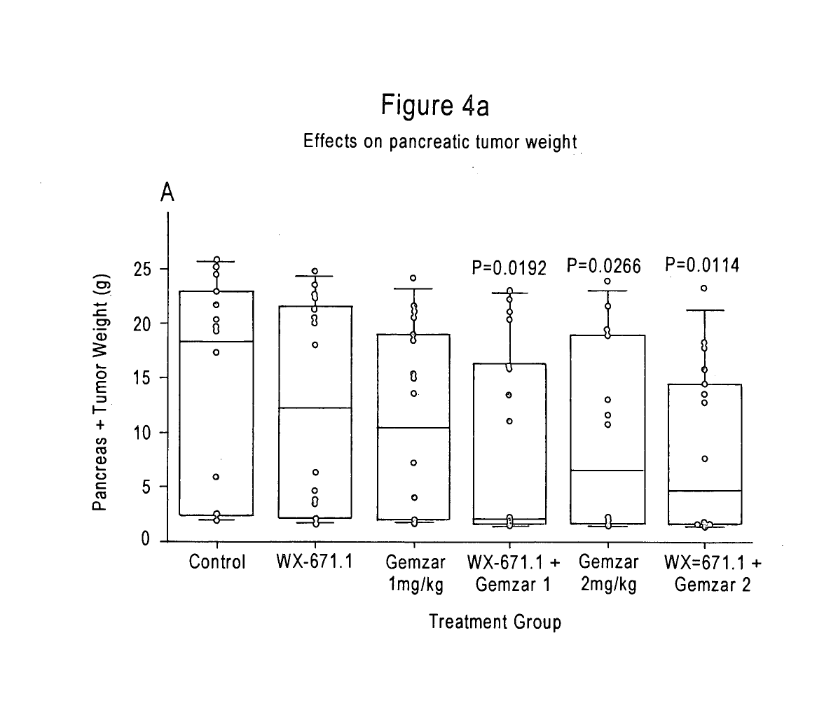

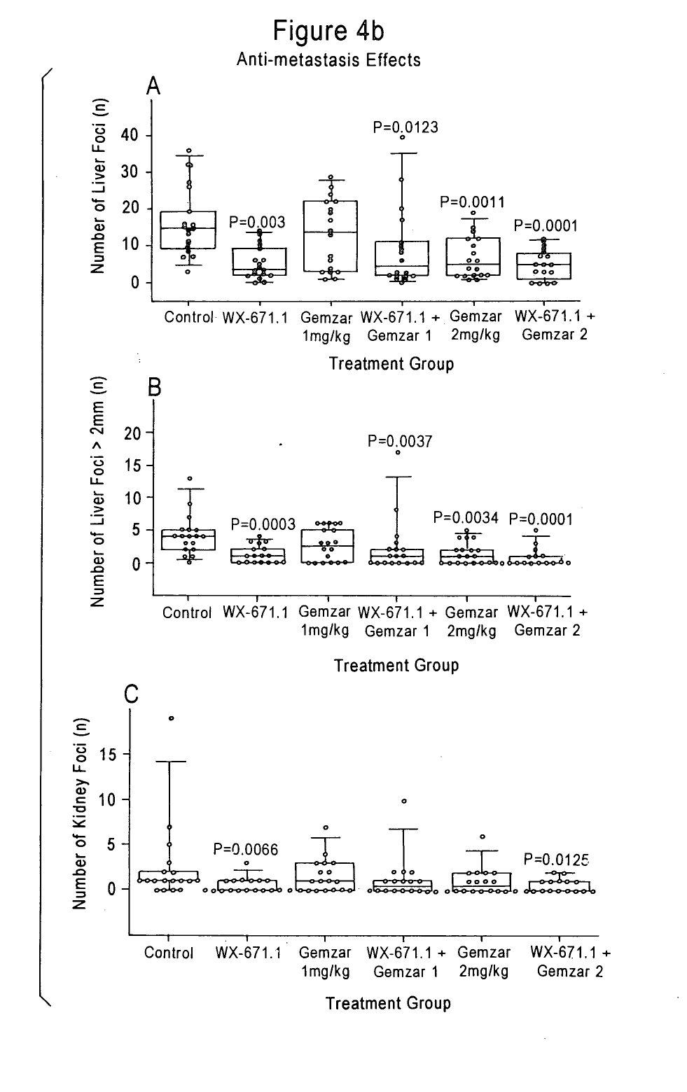

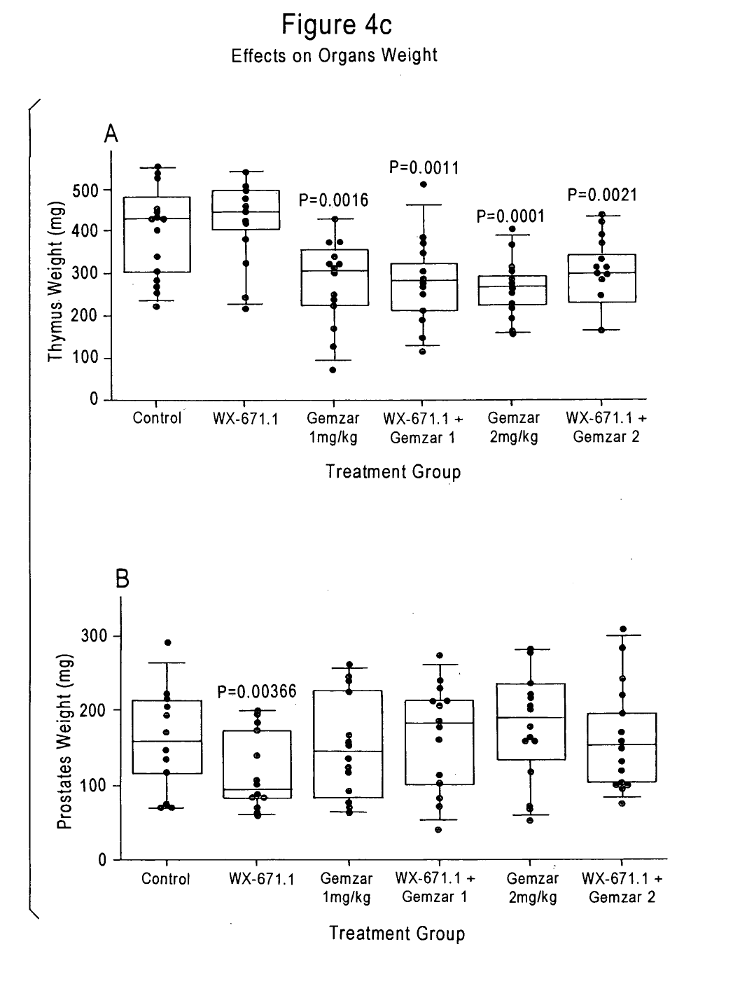

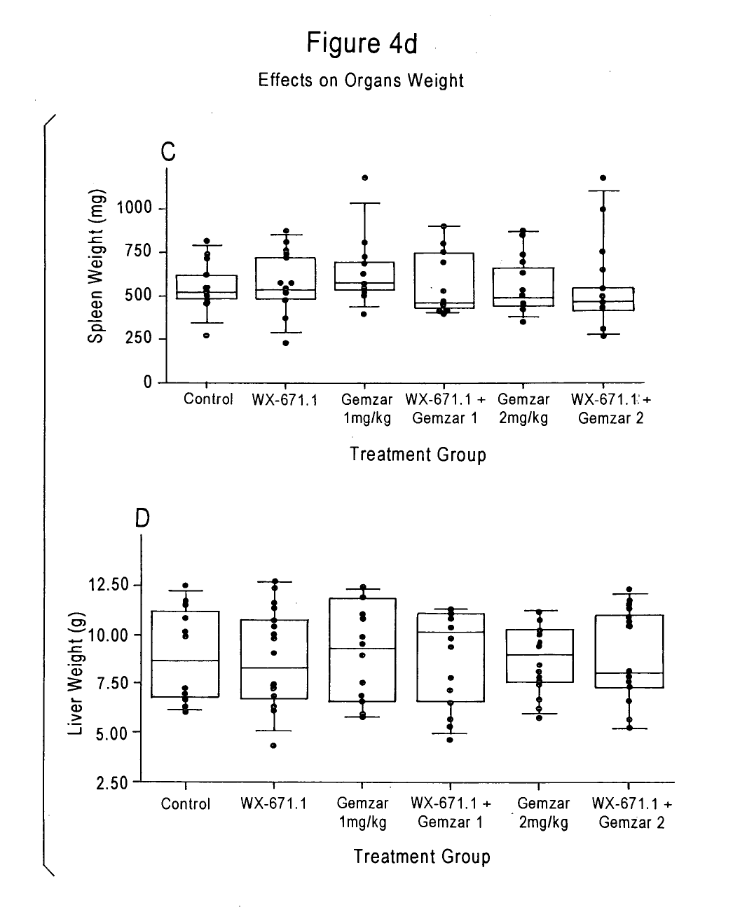

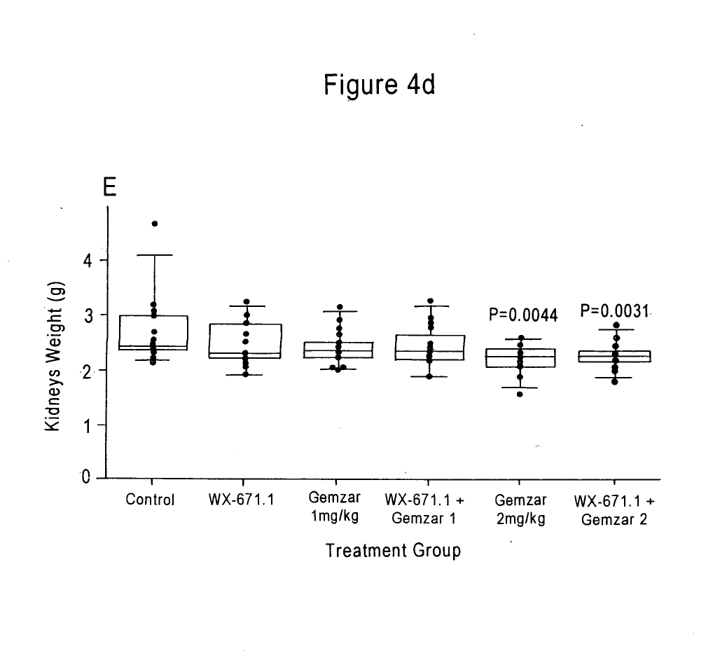

The effects of treatments with WX-671 and gemcitabine on body weight, rat behavior, activity and condition were evaluated twice weekly. The final evaluation was done at the end of the therapy period after all rats had been sacrificed. Graphical evaluations of the endpoints considered, i.e., tumor weight, and the number of macroscopic liver foci, are depicted in the graphs shown in FIGS. 4, 4a, 4b, 4c and 4d.

The combination of gemcitabine with WX-671 resulted in slightly increased inhibition of metastasis spread to the lung and the axillary lymph nodes. In conclusion, an additive effect of the combination therapy with WX-671 and gemcitabine was observed on tumor growth inhibition and on inhibition of liver metastasis<2 mm. The combination therapy was well tolerated at the doses applied. Thus, the additive anti-metastatic effects gained by combining the therapies are achieved without significant enhancement of unwanted side effects.

1. A method of treatment of cancer comprising administering to a patient in need thereof an urokinase inhibitor and a cytotoxic or cytostatic agent.

2. The method of claim 1, wherein the urokinase inhibitor is a compound of the general formula I or II 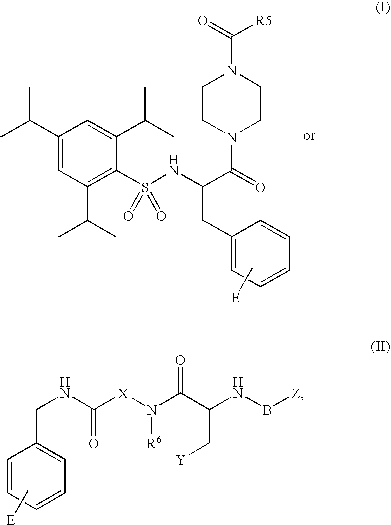
in which E is a group selected from amidine, 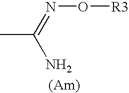
or guanidine 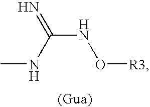
B is —SO₂— or —CO—,
X is —NR¹ or —CHR¹,
Z is —R⁴, —OR⁴ or —NH—R⁴,
Y is —OR² or NHR²,
R¹ is in each case independently —H, —C1-C6-alkyl, —C2-C6-alkenyl or —C2-C6-alkinyl, unsubstituted or substituted,
R² is —H, —OR¹, —COR¹, —COOR¹ or CON(R¹)₂,
R³ is H, C1-C6-alkyl, C2-C6-alkenyl or C2-C6-alkinyl, unsubstituted or substituted, or —COR⁶ or —COOR⁶ or an oligo or polyalkyleneoxy radical, for example with 2-50 —C2-C4-alkyleneoxy, for example ethyleneoxy radicals,
R⁴ is H, —C1-C6-alkyl, —C2-C6-alkenyl or —C2-C6-alkinyl, unsubstituted or substituted, or a cyclic radical,
R⁵ is OR⁶, —N(R⁶)₂, —C1-C6-alkyl, —C2-C6-alkenyl or —C2-C6-alkenyl, unsubstituted or substituted, and
R⁶ is H, —C1-C6-alkyl, —C2-C6-alkenyl or —C2-C6-alkinyl, unsubstituted or substituted, or a cyclic radical,
with each cyclic radical being able to carry one or more substituents, for example selected from the group consisting of —C1-C6-alkyl —OR⁶ (e.g. —OH or —C1-C6-alkoxy), halogen, ═O, —NO₂, —CN, —COOR⁶, —N(R⁶)₂, —NR⁶COR⁶, —NR⁶CON(R⁶)₂ and —OCOR⁶,
and it being possible for each alkyl, alkenyl or alkinyl to be straight-chained or branched and to carry one or more substituents, for example selected from the group consisting of halogen (F, Cl, Br, I), OR⁶, —OCOR⁶, —N(R⁶)₂, —NR⁶COR⁶, —COOR⁶, —NR⁶COR⁶ or a cyclic radical,
and salts or prodrugs of said compounds.

3. The method of claim 1, wherein the urokinase inhibitor is a compound of general formula III 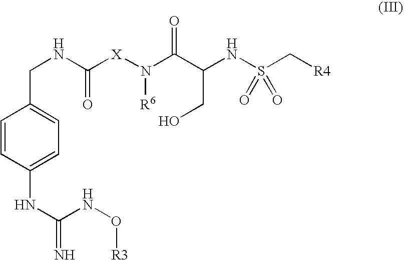
in which X, R¹, R³, R⁴ and R⁶ are defined as in claim 2.

4. The method of claim 1, wherein the urokinase inhibitor is selected from
N-α-(2,4,6-triisopropylphenylsulfonyl)-3-hydroxyamidino-(L)-phenylalanine-4-ethoxycarbonylpiperazde (WX-671),
N-α-(2,4,6-triisopropylphenylsulfonyl)-3-hydroxyamidino-(D)-phenylalanine-ethoxycarbonylpiperazide,
N-α-(2,4,6-triisopropylphenylsulfonyl)-3-hydroxyamidino-(D,L)-phenylalanine-4-ethoxycarbonylpiperazide,
N-α-(2,4,6-triisopropylphenylsulfonyl)-3-hydroxyguanidino-(L)-phenylalanine-4-ethoxycarbonylpiperazide (WX-683),
N-α-(2,4,6-triisopropylphenylsulfonyl)-3-hydroxy-guanidino-(D)-phenylalanine-4-ethoxycarbonylpiperazide,
N-α-(2,4,6-triisopropylphenylsulfonyl)-3-hydroxyguanidino-(D,L)-phenylalanine-4-ethoxycarbonylpiperazide,
N-α-(2,4,6-triisopropylphenylsulfonyl)-3-hydroxy-guanidino-(L)phenylalanine-4-ethylaminocarbonylpiperazide (WX-685),
N-α-(2,4,6-triisopropylphenylsulfonyl)-3-hydroxyguanidino-(D)-phenylalanine-4-ethylaminocarbonylpiperazide,
N-α-(2,4,6-triisopropylphenylsulfonyl)-3-hydroxyguanidino-(D,L)-phenylalanine-4-ethylaminocarbonylpiperazide,
benzylsulfonyl-(D)-Ser-Gly-(4-hydroxyguanidinobenzyl)-amide (WX-678),
4-chlorobenzylsulfonyl-(D)-Ser-N-Me-Ala-(4-hydroxyguanidinobenzyl)amide,
4-chlorobenzylsulfonyl-(D)-Ser-Gly-(4-hydroxyguanidinobenzyl)amide,
10 benzylsulfonyl-(D)-Ser-N-Me-Gly-(4-hydroxyguanidinobenzyl)amide,
4-chlorobenzylsulfonyl-(D)-Ser-Ala-(4-hydroxyguanidinobenzyl)amide,
N-[2-(4-Guanidino-benzenesulfonyl-amino)-ethyl]-3-hydroxy-2-phenylmethane-sulfonylamino-propionamide hydrochloride (WX-568), Bz-SO₂-(D)-Ser-(Aza-Gly)-4-guanidino-benzylamide hydrochloride (WX-544), N-(4-guanidino-benzyl)-2-(3-hydroxy-2-phenylmethane-sulfonylamino-propionylamino)-4-phenyl-butyramide hydrochloride (WX-550), 3-nitrobenzyl-sulfonyl-(D)-Ser-Gly-(4-guanidinobenzyl)amide hydrochloride (WX-C316), benzylsulfonyl-(D)-Ser-N-Me-Gly-(4-guanidinobenzyl)amide (WX-538), N-[(4-guanidino-benzylcarbamoyl)-methyl]-3-hydroxy-2-phenylmethanesulfonylaminopropionamide (WX-508), 4-chlorobenzyl-sulfonyl-(D)-Ser-N-Me-Ala-(4-guanidinobenzyl)amide (WX-582), 4-chloro-benzylsulfonyl-(D)-Ser-Gly-(4-guanidinobenzyl)amide hydrochloride (WX-C340), 3-chlorobenzylsulfonyl-(D)-Ser-Gly-(4-guanidinobenzyl)amide hydrochloride (WX-C318).

5. The method of claim 1, wherein the urokinase inhibitor is N-α-(2,4,6-triisopropylphenylsulfonyl)-3-hydroxyamidino-(L)-phenylalanine-4-ethoxycarbonyl-piperazde (WX-671).

6. The method of claim 1, wherein the urokinase inhibitor is an orally administrable agent.

7. The method of claim 1, wherein the cytotoxic or cytostatic agent is selected from the group consisting of G250 antibody, 5-fluorouracil (5-FU), irinotecan, gemcitabine and capecitabine.

8. The method of claim 1 for the treatment of mammary carcinoma, pancreas carcinoma, colon carcinoma, kidney cancer, lung cancer and head and neck tumors.

9. The method of claim 1 for reducing metastases.
